(19) United States
(12) Patent Application Publication
(10) Pub. No.: US 2007/0249988 A1
(43) Pub. Date: Oct. 25, 2007

(54) Electrotransport Delivery of Nesiritide

(75) Inventors: Rama V. Padmanabhan, Los Altos, CA (US); Joseph Bradley Phipps, Sunnyvale, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: ALZA CORPORATION, MOUNTAIN VIEW, CA (US)

(21) Appl. No.: 11/691,005

(22) Filed: Mar. 26, 2007

Related U.S. Application Data
(60) Provisional application No. 60/794,236, filed on Apr. 21, 2006.

(51) Int. Cl. A61N 1/30

(52) U.S. Cl. 604/20

(57) ABSTRACT

The present invention provides methods and devices for the non-invasive, transdermal administration by electrotransport of nesiritide, or pharmaceutically acceptable nesiritide salts, to patients in need of treatment with nesiritide. The present invention also provides methods for the treatment of congestive heart failure that involve the administration of nesiritide, or pharmaceutically acceptable nesiritide salts, by electrotransport to patients that suffer from congestive heart failure.

### CROSS REFERENCE TO RELATED APPLICATIONS

This application claims the benefit of U.S. application Ser. No. 60/794,236, filed Apr. 21, 2006, which is incorporated herein by reference in its entirety.

### FIELD OF THE INVENTION

The present invention relates to methods and devices for the electrotransport delivery of nesiritide, or pharmaceutically acceptable salts thereof, to patients in need of treatment with nesiritide. The invention further relates to methods of treating congestive heart failure that involve delivery via electrotransport of nesiritide, or pharmaceutically acceptable salts thereof, to patients that suffer from congestive heart failure.

### BACKGROUND OF THE INVENTION

Brain natriuretic peptides (BNPs) have favorable effects on the hemodynamic profile of patients with heart failure, producing a fall in systemic vascular resistance and a mild reduction in arterial pressure (Colucci, W. S., et al., N. Engl. J. Med. 343:246-253 (2000)). The neuroendocrinologic alterations seen after the administration of BNPs include a decrease in aldosterone levels and a mild decrease in plasma renin activity (McGregor, A., et al., J. Clin. Endo. Metab. 70:1103-1107 (1990); Holmes, S. J., et al., J. Clin. Endo. Metab. 76: 91-96 (1993); Yoshimura, M., et al., Circulation 84:1581-1588 (1991)). BNPs inhibit the antinatriuretic effect of angiotensin II and aldosterone on the proximal and distal convoluted tubules. BNPs also increase distal sodium delivery and decrease proximal and distal tubular sodium reabsorption, maintain glomerular filtration rates, and have modest diuretic properties, causing increases in urinary sodium and volume (Marcus, L. S., et al., Circulation 94:3184-3189 (1996)).

Nesiritide is a synthetic recombinant human brain or B-type natriuretic peptide (hBNP) identical to the endogenous peptide released by the ventricle in response to stress, hypertrophy, and volume overload. Nesiritide has 32 amino acids and has a molecular weight of 3466 Da. The cysteine residues at positions 7 and 23 of nesiritide form a disulphide bridge.

Nesiritide displays vasodilatory, natriuretic, neurohormonal and diuretic effects, which make it a nearly ideal drug for the treatment of acute decompensated congestive heart failure (Fonarow G C, Reviews of Cardiovascular Medicine, Vol. 2 Suppl. 2, S32-S35, 2001; G M Keating and K L Goa Drugs 63(1): 47-70, 2003). Congestive heart failure occurs when the heart fails to pump blood adequately, resulting in congestion in pulmonary and systemic circulation and diminished blood flow to tissues (Poole-Wilson, JAMA Mar. 27, 2004).

Patients presenting to the emergency room with acutely decompensated congestive heart failure pose a significant health care problem (G C Fonarow, Reviews in Cardiovascular Medicine 3, Supplement 4, S18-S27, 2002). Such patients are often hemodynamically very unstable, have disabling symptoms of dyspnea, and most require hospitalization. An estimated 5 million people in the United States have congestive heart failure, and each year approximately 990,000 hospital admissions result in congestive heart failure as a primary diagnosis. Two million patients are hospitalized annually in the United States with congestive heart failure as a secondary diagnosis. Congestive heart failure is the most common discharge diagnosis for patients over the age of 65 and is the single largest expense for Medicare.

Oral pharmacotherapeutics are the first line of treatment for ambulatory patients suffering from congestive heart failure, while intravenous strategies are used in hospitalized, acutely decompensated patients. While there have been a number of new drugs introduced that can be taken orally for the treatment of chronic congestive heart failure, limited progress has been made in the management of acute congestive heart failure. This limited progress is due, in part, to the complex regimens that must be followed for the treatment of acute congestive heart failure, with numerous drugs required in varying doses at different times during progression of the disease.

Nesiritide is currently approved as an intravenous dosage form for the treatment of acute decompensated congestive heart failure in a hospital setting. Nesiritide causes hypotension (W S Colucci, J Cardiac Failure Vol. 7 No. 1, 92-100, 2001), and it is therefore administered in settings where blood pressure can be closely monitored to facilitate rapid adjustments in dosing. Nesiritide shows promise, however, for the treatment of acute congestive heart failure in the outpatient or home setting for patients at risk for hospitalization. There is a need for devices and methods that will allow for the safe, non-invasive, continuous infusion-like delivery of nesiritide in an outpatient or home setting.

### SUMMARY OF THE INVENTION

Particular aspects of the present invention relate to methods for the transdermal administration by electrotransport of nesiritide, or a pharmaceutically acceptable salt thereof, to a patient in need of nesiritide that comprise providing a device for the electrotransport delivery of nesiritide and administering nesiritide or a pharmaceutically acceptable nesiritide salt to the patient at a therapeutically effective dose using the device. In certain embodiments of the invention, the device comprises a donor electrode assembly; a counter electrode assembly; and a source of electrical power that is connected to the donor and counter electrode assemblies. In preferred aspects of the invention, the donor electrode assembly comprises a donor reservoir that comprises a matrix that contains nesiritide or a pharmaceutically acceptable nesiritide salt.

Other aspects of the present invention relate to devices for the transdermal administration by electrotransport of nesiritide, or a pharmaceutically acceptable salt thereof, to a patient in need of nesiritide that comprise a donor electrode assembly; a counter electrode assembly; and a source of electrical power that is connected to the donor and counter electrode assemblies. In preferred embodiments of the invention, the donor electrode assembly comprises a donor reservoir that comprises a matrix containing nesiritide or a pharmaceutically acceptable nesiritide salt.

Still further embodiments of the present invention involve methods for the treatment of congestive heart failure that consist essentially of transdermally administering nesiritide, or a pharmaceutically acceptable salt thereof, to a patient suffering from congestive heart failure using an electrotransport device. In preferred aspects of the invention, the electrotransport device comprises a donor electrode assembly; a counter electrode assembly; and a source of electrical power that is connected to the donor and counter electrode assemblies. Preferably, the donor electrode assembly comprises a donor reservoir that comprises a matrix containing nesiritide or a pharmaceutically acceptable nesiritide salt.

### BRIEF DESCRIPTION OF THE DRAWINGS

FIG. 1A depicts the circular dichroic (CD) spectra of unbuffered nesiritide at pH 5.5 and 32° C. under varied conditions.

FIG. 1B depicts the different spectra of nesiritde in 40% TFE, with reference spectra at pH 7.0, cacodylate, 10 mM ionic, 20° C.

FIG. 2 shows the mean flux for in vitro electrotransport of nesiritide over a period of 25 hours. The donor solution was 1 mM or 3 mM nesiritide, pH 7 imidazole, 10 mM ionic. The receptor solution was pH 7 imidazole, 10 mM ionic, 15 mM NaCl, 0.5% DTAB or DTAC. The current used was 100 μA/cm² at 32° C.

FIG. 3 is a histogram depicting the median flux for in vitro electrotransport of nesiritide over a period of 8 hours through fresh human skin. The donor solution was 1 mM or 3 mM nesiritide, pH 7 imidazole, 10 mM ionic. The receptor solution was pH 7 imidazole, 10 mM ionic, 15 mM NaCl, 0.5% DTAB or DTAC. The current used was 100 μA/cm² at 32° C.

FIG. 4 shows the median flux for in vitro electrotransport of nesiritide over a period of 25 hours. The donor solution was 1 mM or 3 mM nesiritide, pH 7 imidazole, 10 mM ionic. The receptor solution was pH 7 imidazole, 10 mM ionic, 15 mM NaCl, 0.5% DTAB or DTAC. The current used was 100 μA/cm² at 32° C.

FIG. 5 shows the in vitro transdermal electrotransport flux of nesiritide across heat-separated human epidermis over time. Cadaver skin was used in the experiments, the current used was 100 μA/cm², 5% nesiritide was used, and the receptor solution was citrate buffered 0.015 M NaCl, pH 5.

FIG. 6A depicts HPLC traces of nesiritide extracted from hydrogel formulations that were not subjected to a current but that were exposed to a synthetic Nyclepore membrane.

FIG. 6B depicts HPLC traces of nesiritide extracted from hydrogel formulations following electrotransport through a synthetic Nuclepore membrane at 100 μA/cm².

FIG. 7A depicts HPLC traces of nesiritide extracted from hydrogel formulations that were not subjected to a current but that were exposed to human epidermis.

FIG. 7B depicts HPLC traces of nesiritide extracted from hydrogel formulations following electrotransport through human epidermis at 100 μA/cm².

FIG. 8 is a perspective exploded view of an electrotransport drug delivery device in accordance with certain aspects of the present invention.

### DETAILED DESCRIPTION OF ILLUSTRATIVE EMBODIMENTS

In particular aspects, the present invention relates to non-invasive, needle-free methods for the delivery across biological tissues of therapeutically meaningful doses of nesiritide or a pharmaceutically acceptable nesiritide salt using electrotransport. In some embodiments of the invention, nesiritide or a pharmaceutically acceptable nesiritide salt is delivered for the treatment of congestive heart failure. Certain aspects of the invention involve the delivery of continuous doses of nesiritide or a pharmaceutically acceptable nesiritide salt, while other aspects involve the delivery of bolus doses of nesiritide or a pharmaceutically acceptable nesiritide salt, intermittent doses of nesiritide or a pharmaceutically acceptable nesiritide salt, or bolus doses of nesiritide or a pharmaceutically acceptable nesiritide salt followed by continuous doses of nesiritide or a pharmaceutically acceptable nesiritide salt. Other aspects of the invention relate to electrotransport devices for the delivery of nesiritide or a pharmaceutically acceptable nesiritide salt that include an electrical power source connected to a sensor that monitors the patient's blood pressure. In such devices, the output of the electrical power source is automatically adjusted in accordance with changes in the patient's blood pressure.

Transdermal electrotransport delivery of nesiritide avoids the drawbacks associated with the oral delivery of nesiritide, which include poor oral bioavailability, variable oral absorption, gastrointestinal degradation, and hepatic first pass effects. In addition, electrotransport delivery of nesiritide provides noninvasive, needle-free, precise, continuous infusion-like dosing that can occur in an outpatient or home setting; it allows the administration of nesiritide to be rapidly terminated; it allows the dosage of nesiritide to be easily adjusted; it reduces the need for hospitalization and its associated costs; it is convenient, which leads to patient compliance; it allows for on-demand dosing; and it allows for feedback controlled dosing.

As used herein, the terms “electrotransport,” “iontophoresis,” and “iontophoretic” refer to the delivery of pharmaceutically active agents (charged, uncharged, or mixtures thereof) through a body surface (such as skin, mucous membrane, eye, or nail) wherein the delivery is at least partially induced or aided by the application of an electric potential. The agent may be delivered by electromigration, electroporation, electroosmosis or any combination thereof. Electromigration (also called iontophoresis) involves the electrically induced transport of charged ions through a body surface. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, i.e., solvent flow induced by electromigration of other ionic species. During the electrotransport process, certain modifications or alterations of the skin may occur such as the formation of transiently existing pores in the skin, also referred to as “electroporation.” Any electrically assisted transport of species enhanced by modifications or alterations to the body surface (e.g., formation of pores in the skin) are also included in the term “electrotransport” as used herein. Thus, as used herein, the terms “electrotransport,” “iontophoresis” and “iontophoretic” refer to (1) the delivery of charged drugs or agents by electromigration, (2) the delivery of uncharged drugs or agents by the process of electroosmosis, (3) the delivery of charged or uncharged drugs by electroporation, (4) the delivery of charged drugs or agents by the combined processes of electromigration and electroosmosis, and/or (5) the delivery of a mixture of charged and uncharged drugs or agents by the combined processes of electromigration and electroosmosis.

In electrotransport devices, at least two electrodes are used. Both of the electrodes are disposed so as to be in intimate electrical contact with some portion of the skin, nails, mucous membrane, or other surface of the body. One electrode, called the “active” or “donor” electrode, is the electrode from which the drug is delivered into the body. The other electrode, called the “counter” or “return” electrode, serves to close the electrical circuit through the body. In conjunction with the patient's skin, the circuit is completed by connection of the electrodes to a source of electrical power, e.g., a battery, and usually to circuitry capable of controlling current passing through the device. If the ionic substance to be driven into the body is positively charged, then the positive electrode (the anode) will be the donor electrode and the negative electrode (the cathode) will serve as the counter electrode, completing the circuit. If the ionic substance to be delivered is negatively charged, then the cathodic electrode will be the donor electrode and the anodic electrode will be the counter electrode. Both the anode and the cathode can be donor electrodes if both anionic and cationic therapeutic agent ions are to be delivered, or if an uncharged therapeutic agent is to be delivered.

Electrotransport devices additionally require a reservoir or source of the pharmaceutically active agent that is to be delivered or introduced into the body. Examples of donor reservoirs include a pouch or cavity, a porous sponge or pad, and a hydrophilic polymer or gel matrix. Such drug reservoirs can be part of a donor electrode assembly, and are connected to, and positioned between, the donor electrode of the electrotransport device and the body surface, to provide a fixed or renewable source of one or more desired species or agents.

Electrotransport devices are powered by an electrical power source such as one or more batteries. Typically, at any one time, one pole of the power source is electrically connected to the donor electrode, while the opposite pole is electrically connected to the counter electrode. Since it has been shown that the rate of electrotransport drug delivery is approximately proportional to the electric current applied by the device, many electrotransport devices typically have an electrical controller that controls the voltage and/or current applied through the electrodes, thereby regulating the rate of drug delivery. These control circuits use a variety of electrical components to control the electrical signal, i.e., the amplitude, polarity, timing, waveform shape, etc. of the electric current and/or voltage, supplied by the power source. U.S. Pat. No. 5,047,007 to McNichols, et al., which is hereby incorporated by reference in its entirety, discloses several suitable parameters and characteristics.

An electrotransport device or system, with its donor and counter electrodes, may be thought of as an electrochemical cell having two electrodes, each electrode having an associated half cell reaction, between which electrical current flows. Electrical current flowing through the conductive (e.g., metal) portions of the circuit is carried by electrons (electronic conduction), while current flowing through the liquid-containing portions of the device (i.e., the drug reservoir in the donor electrode, the electrolyte reservoir in the counter electrode, and the patient's body) is carried by ions (ionic conduction). Current is transferred from the metal portions to the liquid phase by means of oxidation and reduction charge transfer reactions that typically occur at the interface between the metal portion (e.g., a metal electrode) and the liquid phase (e.g., the drug solution). A detailed description of the electrochemical oxidation and reduction charge transfer reactions of the type involved in electrically assisted drug transport can be found in electrochemistry texts such as J. S. Newman, Electrochemical Systems (Prentice Hall, 1973) and A. J. Bard and L. R. Faulkner, Electrochemical Methods, Fundamentals and Applications (John Wiley & Sons, 1980).

As used herein, the term “patient” refers to a mammal, preferably a human.

The term “therapeutically effective dose,” as used herein, refers to the amount of nesiritide or a pharmaceutically acceptable nesiritide salt that, when administered to a patient, is effective to at least partially treat a condition from which the patient suffers. Such conditions include, but are not limited to, congestive heart failure.

The terms “treat” or “treating,” as used herein, refer to partially or completely alleviating, inhibiting, preventing, ameliorating and/or relieving a condition from which a patient suffers.

The terms “suffer” or “suffering” as used herein, refer to one or more conditions that a patient has been diagnosed with, or is suspected to have.

The term “pharmaceutically acceptable salt” refers to salts of nesiritide that retain the biological effectiveness and properties of nesiritide, and that are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts can be prepared from inorganic and organic acids. Salts derived from inorganic acids include, for example, hydrochloric acid, hydrobromic acid, sulfuric acid, nitric acid, phosphoric acid, and the like. Salts derived from organic acids include, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the terms “transdermal administration” and “transdermally administering” refer to the delivery of a substance or agent by passage into and through the skin, nails, mucous membrane, or other surface of the body.

As used herein, the term “matrix” refers to a porous, composite, solid, or semi-solid substance, such as, for example, a polymeric material or a gel, that has pores or spaces sufficiently large for nesiritide or a pharmaceutically acceptable nesiritide salt to populate. The matrix serves as a repository in which nesiritide or a pharmaceutically acceptable nesiritide salt is contained.

Particular aspects of the present invention relate to methods and devices for the transdermal administration by electrotransport of nesiritide, or a pharmaceutically acceptable nesiritide salt, to patients in need of treatment with nesiritide. In preferred embodiments of the invention, the methods comprise providing a device for the electrotransport delivery of nesiritide or a pharmaceutically acceptable nesiritide salt that comprises a donor electrode assembly; a counter electrode assembly; and a source of electrical power that is connected to the donor and counter electrode assemblies; and administering the nesiritide or pharmaceutically acceptable nesiritide salt to the patient at a therapeutically effective dose using the device. In preferred embodiments of the invention, the donor electrode assembly comprises a donor reservoir that comprises a matrix containing nesiritide or a pharmaceutically acceptable nesiritide salt.

Other aspects of the invention relate to methods for treating congestive heart failure that involve transdermally administering nesiritide, or a pharmaceutically acceptable nesiritide salt, to patients suffering from congestive heart failure. In preferred embodiments of the invention, such methods involve the use of an electrotransport device comprising a donor electrode assembly, a counter electrode assembly, and a source of electrical power that is connected to the donor and counter electrode assemblies. In certain embodiments of the invention, the donor electrode assembly comprises a donor reservoir that comprises a matrix containing nesiritide or a pharmaceutically acceptable nesiritide salt.

Nesiritide or pharmaceutically acceptable nesiritide salts can be administered to patients via electrotransport according to certain embodiments of the invention using any of various possible dosing regimes, which include, for example, continuous dosing, intermittent dosing, bolus dosing, bolus dosing followed by continuous dosing, and combinations thereof. A useful therapeutic infusion rate for nesiritide is about 0.005 to 0.05 μg/kg/min, which corresponds to a daily dose of about 0.5 to 5 mg, respectively, as described in the Physician's Desk Reference, 2006, for Natrecor® (nesiritide for injection). In preferred embodiments of the invention, nesiritide or a pharmaceutically acceptable nesiritide salt is administered continuously at a dose of 0.500 μg/kg/min to 0.05 μg/kg/min. In other preferred embodiments, nesiride is administered in a bolus dose of 2 μg/kg/min followed by a continuous dose of 0.01 μg/kg/min. Maintenance of accurate dosing of nesiritide is critical because underdosing does not provide the necessary vasodilatory and natriuretic properties, while overdosing increases the risk of hypotension.

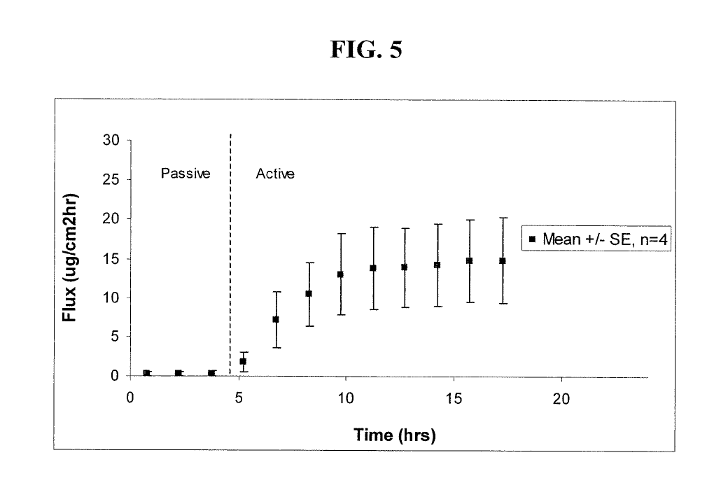

Suitable nesiritide flux rates can be achieved by selecting appropriate electrotransport conditions. As shown in FIG. 5, the electrotransport devices and methods of the invention provide an accurate correlation between the applied current and steady state agent flux in vitro. The data shown in the figure were obtained using the transdermal delivery of nesiritide with a silver donor electrode at the anode and a silver chloride counter electrode at the cathode. As demonstrated, there is a linear correlation between the magnitude of applied current to the steady state flux with an in vitro transport efficiency of about 0.14 mg/mAhr. The tests were performed on heat separated human epidermis.

Given the linear relationship between applied current and nesiritide flux, one of skill in the art can select appropriate electrotransport conditions to achieve therapeutic dosing of nesiritide. The following table provides a range of electrotransport conditions suitable to provide a therapeutic dose of nesiritide shown below as daily dose in mg estimated for a person weighing 70 kg. The current and area estimates are provided assuming a transport efficiency of 0.14 mg/mAh and an operating current density of 0.1 mA/cm².

| Infusion Rate | Daily Dose | Current | Area |
|---|---|---|---|
| (μg/kg/min) | (mg) | (mA) | (cm²) |
| 0.005 | 0.5 | 0.15 | 1.5 |
| 0.01 | 1.0 | 0.3 | 3 |
| 0.02 | 2.0 | 0.6 | 6 |
| 0.03 | 3.0 | 0.9 | 9 |
| 0.04 | 4.0 | 1.2 | 12 |
| 0.05 | 5.0 | 1.5 | 15 |

Nesiritide was administered to hairless guinea pigs, and the guinea pigs were monitored for skin irritation. The irritation at both 0.1 mA/cm² and 0.2 mA/cm² was characterized as mild, indicating that the electrotransport conditions suitable for maintaining a therapeutic plasma concentration of nesiritide will not cause significant discomfort and can be expected to be acceptable for the patient.

The use of a direct current represents the most straightforward embodiment of the methods for delivering nesiritide or pharmaceutically acceptable nesiritide salts via electrotransport. The use of a constant direct current signal typically provides a very linear relationship between the applied current density and the flux of nesiritide, and, as discussed above, does not cause significant skin irritation. Alternative electrotransport conditions can be employed, however. For example, pulsed current, alternating reverse polarity or time-varying, on-off current patterns may be suitable to prevent or minimize skin irritation if prolonged direct current delivery at a single location is undesirable. The electrotransport delivery devices of embodiments of the invention can utilize any suitable electrical circuits to perform a number of functions. Such circuits include pulsing circuits for delivering a pulsed current, timing circuits for delivering nesiritide or a pharmaceutically acceptable nesiritide salt over predetermined timing and dosing regimens, feedback regulating circuits for delivering nesiritide or a pharmaceutically acceptable nesiritide salt in response to a sensed physical parameter, and polarity controlling circuits for periodically reversing the polarity of the electrodes. See for example, Tapper, et al., U.S. Pat. No. 4,340,047; Lattin, U.S. Pat. No. 4,456,012; Jacobsen, U.S. Pat. No. 4,141,359; and Lattin, et al., U.S. Pat. No. 4,406,658.

Certain embodiments of the invention can thus suitably utilize a pulsed (square wave) current. Duty cycle is the ratio of the “on” time interval to the period of time of one cycle (i.e., the ratio of the pulse-duration time to the pulse-period) and is usually expressed as a percentage. For example, if a device is “on” for 500 ms of a 1 sec cycle, then the device is operating in a 50% duty cycle. The generated load current pattern makes adjustments to the load current either by changing the magnitude or by changing the duty cycle of the pulse. For example, an average current of 0-0.05 mA/cm², 10% duty cycle pulse is 0.005 mA/cm². For the purpose of this embodiment, it is stipulated that the frequency is less than 100 Hz. Doubling the preceding average current is accomplished by increasing the load current to 0-0.1 mA/cm² while keeping the duty cycle constant at 10%, or doubling the duty cycle to 20% while maintaining the load current at 0-0.05 mA/cm². (Note that these relationships are approximations). If otherwise modulated current is used, the load current can be changed by changing the shape of the waveform. The total time of current application could also be adjusted in order to provide a desired agent delivery rate, particularly in on-demand delivery applications.

Modifying the duty cycle of the pulses thus increases or decreases the amount of nesiritide or pharmaceutically acceptable nesiritide salt delivered. In this practice of the invention, the magnitude of the current pulses is selected in view of the known area of the surface from which nesiritide or a pharmaceutically acceptable nesiritide salt is delivered, thereby defining a fixed and known current density (i.e., the ratio of current to the area from which current flows).

As discussed in U.S. Pat. No. 5,983,130, which is hereby incorporated by reference in its entirety, enhanced nesiritide or pharmaceutically acceptable nesiritide salt delivery can be achieved by applying a current density to a body site above a critical level. Once it has been determined that a specific maximum current for a given anode surface area will provide enhanced efficiency of delivery, by increasing or decreasing the duty cycle, the amount of nesiritide or pharmaceutically acceptable nesiritide salt delivered at the high efficiency state can be increased or decreased without causing the maximum applied current density to change. In choosing the parameters of electrotransport using this approach, the amplitude of the current pulses is selected so that the resulting current density transforms the skin into the high efficiency transfer state and the duty cycle of the current pulses is altered to adjust the agent delivery rate. Alternatively, the pulsing frequency of a pulsed current waveform is adjusted to control the overall quantity of drug delivered while maintaining current density at or above the level which transforms the skin into the high efficiency state.

Another suitable type of electrotransport delivery may be characterized as alternating reverse polarity. An example of such a system is described in U.S. Pat. No. 4,406,658, which is hereby incorporated by reference in its entirety. Generally, an ionic species is used to trigger a conversion in the skin to a more permeable state, which allows more efficient agent transfer. As an example, such a system would first drive the anionic drug counter ion from the donor reservoir and the cationic substance from the counter reservoir for the time required to convert the skin to a high efficiency state and then reverse polarity, thereby moving the drug cation into the skin.

It may be desirable to configure the electrotransport transdermal delivery device of embodiments of the invention to be suited to the desired application. For example, a device configured for use in a hospital or clinic may consist of a controller or current source capable of delivering a wide array of dosing levels. As such, the hospital use system can be used to titrate the dosage to obtain and maintain the desired plasma concentration of the nesiritide or pharmaceutically acceptable nesiritide salt. Alternatively, a device configured for individual, independent use by a patient should deliver a single dose that has been determined to be therapeutically effective. Ideally, such a system should require minimal user intervention.

The electrotransport devices and methods of the invention can also be used in a feedback manner to create a closed loop. Specifically, interfacing the electrotransport devices of the invention with a blood pressure monitoring device allows nesiritide flux to be controlled to maintain optimal blood pressure. Information from such monitors can therefore be used to automatically adjust electrotransport conditions to vary the flux of the nesiritide or pharmaceutically acceptable nesiritide salt, and, thus, maintain plasma concentrations of nesiritide or pharmaceutically acceptable nesiritide salt at therapeutically desired levels. Preferred embodiments of the invention thus relate to electrotransport devices that comprise a sensor that monitors the patient's blood pressure, and the output of the electrical power source of the devices is automatically adjusted in accordance with changes in the patient's blood pressure.

While the present invention is not limited to any particular electrotransport device, preferred electrotransport devices allow the patient to self-administer nesiritide or a pharmaceutically acceptable nesiritide salt.

In certain embodiments of the invention, the electrotransport device used for administering nesiritide or a pharmaceutically acceptable nesiritide salt comprises a donor electrode assembly that comprises a donor reservoir that comprises a matrix containing nesiritide or a pharmaceutically acceptable nesiritide salt. The donor reservoir can be any material adapted to absorb and hold a sufficient quantity of liquid therein in order to permit transport of nesiritide or a pharmaceutically acceptable nesiritide salt by electrotransport. The reservoir can be comprised of essentially any suitable synthetic or naturally-occurring polymeric material. The reservoir can be composed, at least in part, of a soluble hydrophilic polymer material, or can be a solid polymer composed, at least in part, of an insoluble hydrophilic polymer. Insoluble hydrophilic polymer reservoirs may be preferred for structural reasons over soluble hydrophilic polymers. The reservoir polymer can be formed in situ, or the polymers can be prefabricated and sorbed with the components from solutions as is the case with cellulose, woven fiber pads, and sponges.

The donor reservoir can alternately be a gel matrix structure wherein the gel is formed of a hydrophilic polymer that is swellable or soluble in water. Such polymers can be blended with the components in any ratio, and represent from a few to about 50 wt % of the reservoir. The polymers can be linear or cross-linked.

Suitable hydrophilic polymers include co-polyesters such as HYTREL® (DuPont De Nemours & Co., Wilmington, Del.), polyvinylpyrrolidones, polyvinyl alcohol, polyethylene oxides such as POLYOX (Union Carbide Corp.), CARBOPOL (BF Goodrich of Akron, Ohio), blends of polyoxyethylene or polyethylene glycols with polyacrylic acid such as POLYOX® blended with CARBOPOL®, polyacrylamide, KLUCEL®, cross-linked dextran such as SEPHADEX® (Pharmacia Fine Chemicals, AB, Uppsala, Sweden), WATER LOCK® (Grain Processing Corp., Muscatine, Iowa) which is a starch-graft-poly(sodium acrylate-co-acrylamide) polymer, cellulose derivatives such as hydroxyethyl cellulose, hydroxypropylmethylcellulose, low-substituted hydroxypropylcellulose, and cross-linked Na-carboxymethylcellulose such as Ac-Di-Sol (FMC Corp., Philadelphia, Pa.), hydrogels such as polyhydroxylethyl methacrylate (National Patent Development Corp.), natural gums, chitosan, pectin, starch, guar gum, locust bean gum, and the like, along with blends thereof. The noted list is merely exemplary of the materials suited for use in this invention, and other suitable hydrophilic polymers can be found in Scott, J. R., & Roff, W. J., Handbook of Common Polymers, CRC Press (1971), the pertinent portions of which being hereby incorporated by reference.

Optionally, a hydrophobic polymer may also be present, to improve the structural integrity of the reservoir. Preferably the hydrophobic polymer is heat fusible, in order to enhance the lamination to adjacent layers. Suitable hydrophobic polymers include, but are not limited to, polyisobutylenes, polyethylene, polypropylene, polyisoprenes and polyalkenes, rubbers, copolymers such as KRATON®, polyvinylacetate, ethylene vinyl acetate copolymers, polyamides such as nylons, polyurethanes, polyvinylchloride, acrylic or methacrylic resins such as polymers of esters of acrylic or methacrylic acid with alcohols such as n-butanol, 1-methyl pentanol, 2-methyl pentanol, 3-methyl pentanol, 2-ethyl butanol isooctanol, n-decanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tert-butylacrylamide, itaconic acid, N-branched alkyl maleamic acids, wherein the alkyl group has 10-24 carbon atoms, glycol diacrylates, and blends thereof. Most of the above-mentioned hydrophobic polymers are heat fusible; however, the materials used in the cathodic reservoir should be selected so that they are compatible with the cetylpyridinium salt.

In addition, the donor reservoir can be a substantially non-hydrated, dry matrix. Dry matrices include matrices that require reconstitution or hydration before use, such as, for example, polyurethane-based Tecogel and HEC (hydroxyethyl cellulose)/Carbopol. Dry matrices also include substantially solvent-free ion-conducting polymer electrolytes that do not require hydration before use, such as, for example, polyethylene oxide, polysiloxanes having a hydrophilic side chain, polyphosphazenes having a hydrophilic side chain, polyethylene succinate, and polyacrylonitrile.

In certain preferred embodiments of the invention, the donor reservoir formulation for transdermally delivering nesiritide or a pharmaceutically acceptable nesiritide salt by electrotransport is comprised of an aqueous solution of a water-soluble pharmaceutically acceptable nesiritide salt. Suitable pharmaceutically acceptable salts of nesiritide include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate, and sulfonate.

The nesiritide or pharmaceutically acceptable nesiritide salt is present in the donor reservoir in an amount sufficient to deliver the above-described doses transdermally by electrotransport over a desired period of time. The nesiritide or pharmaceutically acceptable nesiritide salt typically comprises about 1 to 10 weight % of the donor reservoir formulation (including the weight of the polymeric matrix) on a fully hydrated basis (if a hydrated matrix is being used), and more preferably about 1 to 5 weight % of the donor reservoir formulation on a fully hydrated basis.

Nesiritide and pharmaceutically acceptable nesiritide salts can be formulated for electrotransport by adding one or more of the following ingredients to an aqueous solution of nesiritide or a pharmaceutically acceptable nesiritide salt: preservatives, co-solvents, antioxidants and radical scavengers, chelators, or buffering agents.

Suitable preservatives include, for example, sorbic acid, benzoic acid, benzyl alcohol, the parabens such as propylparaben, ethylparaben, butylparaben, methylparaben, benzylparaben, isobutylparaben, phenoxyethanol, ethanol, Rohm & Haas's Kathon CG®, Dowisil 200, chlorhexidine, Triclosan, Germall, Bronopol, Monolaurin, cetylpyridinium chloride, benzalkonium chloride, sodium metabisulfite, Acticare, dehydroacetic acid, o-phenylphenol, sodium bisulfite, dichlorophen, salts of any of the above compounds, and mixtures of any of the above compounds. Preferred preservatives include benzyl alcohol, benzoic acid, sorbic acid, the parabens, propylparaben, methylparaben, phenoxyethanol, Triclosan, Germall II, Bronopol, Monolaurin, Kathon CG, salts of any of these preservatives, and mixtures of any of these compounds. The following compounds may be required to further enhance the efficacy of the preservative: ethylenediaminetetraacetic acid (EDTA), propylene glycol, or ethanol.

Suitable co-solvents include, but are not limited to, ethanol, propylene glycol and polyethylene glycol.

Anti-oxidants/radical scavengers s include ascorbic acid (vitamin C) and it salts, tocopherol (vitamin E) its salts and esters, esters of tocopherol, butylated hydroxy benzoic acids and their salts, gallic acid and its esters, uric acid and its salts and esters, and sorbic acid and its salts.

Suitable chelating agents include, for example, ethyenediaminetetraacetic acide (EDTA).

Preferred buffers include, for example, dipeptide buffers. Dipeptide buffers comprise a polypeptidic chain of two to five amino acids, and have an isoelectric pH at which the dipeptide carries no net charge. The aqueous solution has a pH which is within about 1.0 pH unit of the isoelectric pH. Preferably, the dipeptide has at least two pKa's that are separated by no more than about 3.5 pH units. Most preferably, the isoelectric pH of the dipeptide is between about 3 and 10. The concentration of the dipeptide buffer in the solution is preferably at least about 10 mM. The dipeptide buffer is preferably selected from the group consisting of Asp-Asp, Gly-Asp, Asp-His, Glu-His, His-Glu, His-Asp, Glu-Arg, Glu-Lys, Arg-Glu, Lys-Glu, Arg-Asp, Lys-Asp, His-Gly, His-Ala, His-Asn, His-Citruline, His-Gin, His-Hydroxyproline, His-Isoleucine, His-Leu, His-Met, His-Phe, His-Pro, His-Ser, His-Thr, His-Trp, His-Tyr, His-Val, Asn-His, Thr-His, Try-His, Gin-His, Phe-His, Ser-His, Citruline-His, Trp-His, Met-His, Val-His, His-His, Isoleucine-His, Hydroxyproline-His, Leu-His, Ala-His, Gly-His, Beta-Alanylhistidine, Pro-His, Camosine, Anserine, Tyr-Arg, Hydroxylysine-His, His-Hydroxytlysine, Ornithine-His, His-Lys, His-Ornithine and Lys-His. A particularly preferred dipeptide buffer is Gly-His.

The nesiritide and pharmaceutically acceptable nesiritide salt formulations are used in an electrotransport device such as described hereinafter. A suitable electrotransport device includes an anodic donor electrode, preferably comprised of silver, and a cathodic counter electrode, preferably comprised of silver chloride. The donor electrode is in electrical contact with the donor reservoir containing the aqueous solution of nesiritide or a pharmaceutically acceptable nesiritide salt. The counter reservoir contains a (e.g., aqueous) solution of a biocompatible electrolyte, such as citrate buffered saline. The anodic and cathodic reservoirs preferably each have a skin contact area of about 1 to 5 cm² and more preferably about 2 to 3 cm². The anodic and cathodic reservoirs preferably have a thickness of about 0.05 to 0.25 cm, and more preferably about 0.15 cm. The applied electrotransport current is about 150 μA to about 240 μA. Most preferably, the applied electrotransport current is substantially constant DC current during the dosing interval.

The cathodic electrode and the anodic electrode are comprised of electrically conductive material such as a metal. For example, the electrodes can be formed from a metal foil, a metal screen, or metal deposited or painted on a suitable backing, or by calendaring, film evaporating, or mixing the electrically conductive material in a polymer binder matrix. Examples of suitable electrically conductive materials include carbon, graphite, silver, zinc, aluminum, platinum, stainless steel, gold and titanium. For example, as noted above, the anodic electrode can be composed of silver, which is also electrochemically oxidizable. The cathodic electrode can be composed of carbon and electrochemically reducible silver chloride. Silver is preferred over other metals because of its relatively low toxicity to mammals. Silver chloride is preferred because the electrochemical reduction reaction occurring at the cathode (AgCl+e⁻Ag+Cl⁻) produces chloride ions which are prevalent in, and non-toxic to, most animals.

The source of electrical power electrically connected to the anode and the cathode can be of any variety. For instance, if the counter and donor electrodes are of dissimilar metals or have different half cell reactions, it is possible for the system to generate its own electrical power. Typical materials that provide a galvanic couple include a zinc donor electrode and a silver chloride counter electrode. Such a combination will produce a potential of about one volt. When a galvanic couple is used, the donor electrode and counter electrode are integral portions of the power generating process. Such a galvanic couple powered system, absent some controlling means, activates automatically when body tissue and/or fluids form a complete circuit with the system. There exist numerous other examples of galvanic couple systems potentially useful in the present invention.

In some instances it may be necessary to augment the power supplied by the galvanic electrode couple, which may be accomplished with the use of a separate electrical power source. Such a power source is typically a battery or plurality of batteries, connected in series or in parallel, and positioned between the cathodic electrode and the anodic electrode such that one electrode is connected to one pole of the power source and the other electrode is connected to the opposite pole. Commonly, one or more 3 volt button cell batteries are suitable to power electrotransport devices. A preferred battery is a 3 volt lithium button cell battery.

The power source can include electronic circuitry for controlling the operation of the electrotransport device. Thus, the power source can include circuitry designed to permit the patient to manually turn the system on and off, such as with an on demand medication regime, or to turn the system on and off at some desired periodicity, for example, to match the natural or circadian patterns of the body. In addition, the control means can limit the number of doses that can be administered to the patient. A relatively simple controller or microprocessor could control the current as a function of time or could generate complex current waveforms such as pulses or sinusoidal waves. The control circuitry can also include a biosensor and some type of feedback system that monitors biosignals, provides an assessment of therapy, and adjusts the drug delivery accordingly.

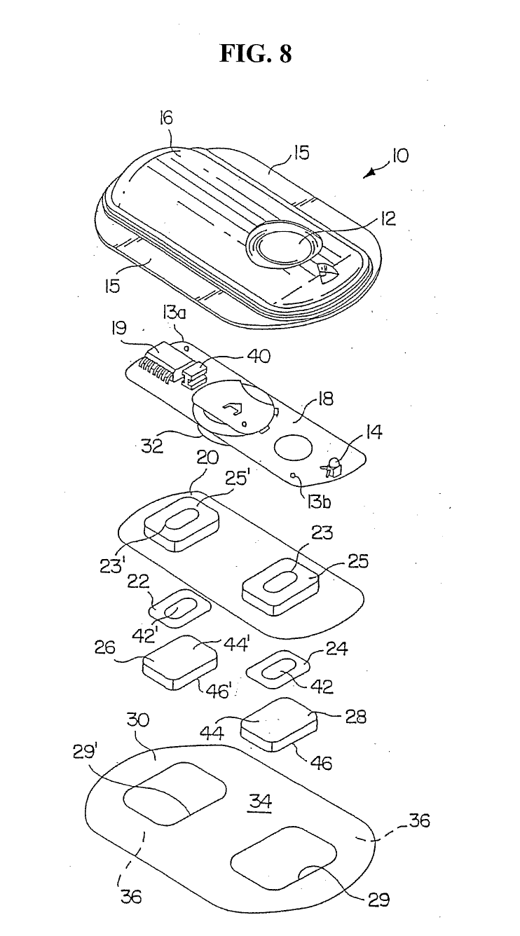

Reference is now made to FIG. 8, which depicts an exemplary electrotransport device that can be used in accordance with the present invention. FIG. 8 shows a perspective exploded view of an electrotransport device 10 having an activation switch in the form of a push button switch 12 and a display in the form of a light emitting diode (LED) 14. Device 10 comprises an upper housing 16, a circuit board assembly 18, a lower housing 20, anode electrode 22, cathode electrode 24, anode reservoir 26, cathode reservoir 28 and skin-compatible adhesive 30. Upper housing 16 has lateral wings 15 that assist in holding device 10 on a patient's skin. Upper housing 16 is preferably composed of an injection moldable elastomer (e.g., ethylene vinyl acetate). Printed circuit board assembly 18 comprises an integrated circuit 19 coupled to discrete electrical components 40 and battery 32. Circuit board assembly 18 is attached to housing 16 by posts (not shown in FIG. 8) passing through openings 13a and 13b, the ends of the posts being heated/melted in order to heat stake the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of circuit board assembly 18 is a battery 32, which is preferably a button cell battery and most preferably a lithium cell. Other types of batteries may also be employed to power device 10.

The circuit outputs (not shown in FIG. 8) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′,44 of reservoirs 26 and 28. The bottom sides 46′,46 of reservoirs 26,28 contact the patient's skin through the openings 29′,29 in adhesive 30. Upon depression of push button switch 12, the electronic circuitry on circuit board assembly 18 delivers a predetermined DC current to the electrodes/reservoirs 22,26 and 24,28 for a delivery interval of predetermined length, e.g., about 10 minutes. Preferably, the device transmits to the user a visual and/or audible confirmation of the onset of the drug delivery, or bolus, interval by means of LED 14 becoming lit and/or an audible sound signal from, e.g., a “beeper”. Nesiritide or a pharmaceutically acceptable nesiritide salt is then delivered through the patient's skin, e.g., on the arm, for the predetermined (e.g., 10 minute) delivery interval. In practice, a user receives feedback as to the onset of the drug delivery interval by visual (LED 14 becomes lit) and/or audible signals (a beep from the “beeper”).

Anodic electrode 22 is preferably comprised of silver and cathodic electrode 24 is preferably comprised of silver chloride. Both reservoirs 26 and 28 are preferably comprised of polymer hydrogel materials as described herein. Electrodes 22, 24 and reservoirs 26, 28 are retained by lower housing 20. For nesiritide and pharmaceutically acceptable nesiritide salts, the anodic reservoir 26 is the “donor” reservoir which contains the drug and the cathodic reservoir 28 contains a biocompatible electrolyte.

The push button switch 12, the electronic circuitry on circuit board assembly 18 and the battery 32 are adhesively “sealed” between upper housing 16 and lower housing 20. Upper housing 16 is preferably composed of rubber or other elastomeric material. Lower housing 20 is preferably composed of a plastic or elastomeric sheet material (e.g., polyethylene) which can be easily molded to form depressions 25,25′ and cut to form openings 23,23′. The assembled device 10 is preferably water resistant (i.e., splash proof), and is most preferably waterproof. The system has a low profile that easily conforms to the body thereby allowing freedom of movement at, and around, the wearing site. The anode/drug reservoir 26 and the cathode/salt reservoir 28 are located on the skin-contacting side of device 10 and are sufficiently separated to prevent accidental electrical shorting during normal handling and use.

The device 10 adheres to the patient's body surface (e.g., skin) by means of a peripheral adhesive 30 which has upper side 34 and body-contacting side 36. The adhesive side 36 has adhesive properties which assures that the device 10 remains in place on the body during normal user activity, and yet permits reasonable removal after the predetermined (e.g., 24-hour) wear period. Upper adhesive side 34 adheres to lower housing 20 and retains the electrodes and drug reservoirs within housing depressions 25,25′ as well as retains lower housing 20 attached to upper housing 16.

The push button switch 12 is located on the top side of device 10 and is easily actuated through clothing. A double press of the push button switch 12 within a short period of time, e.g., three seconds, is preferably used to activate the device 10 for delivery of drug, thereby minimizing the likelihood of inadvertent actuation of the device 10.

Upon switch activation an audible alarm signals the start of drug delivery, at which time the circuit supplies a predetermined level of DC current to the electrodes/reservoirs for a predetermined (e.g., 10 minute) delivery interval. The LED 14 remains “on” throughout the delivery interval indicating that the device 10 is in an active drug delivery mode. The battery preferably has sufficient capacity to continuously power the device 10 at the predetermined level of DC current for the entire (e.g., 24 hour) wearing period.

The following examples are illustrative of certain embodiments of the invention and should not be considered to limit the scope of the invention.

### EXAMPLE 1

### Analysis of Nesiritide by Isocratic Hydrophobic Interaction Chromatography (HIC)

Nesiritide was analyzed by isocratic hydrophobic interaction chromatography (HIC) using the conditions set forth in the table below to estimate the peptide's hydrophobicity.

| Description | Parameter |
|---|---|
| Column: | Pharmacia Superdex Peptide HR 10/30 |
| Flowrate: | 0.5 mL/min |
| Detection: | absorbance at 214 nm, 258 nm |
| Temperature: | column, 32° C.; autosampler, 4° C. |
| Injection: | 50 mL |
| Solvent: | pH 7.0: imidazole 10 mM ionic |
| Retention: | BNP: 4.4 ± 0.1 min; Vₒ: 1.9 ± 0.1 min; 4.9 ± 0.1 min |
| System: | ThermoSeparations |

### Nesiritide demonstrated high measured hydrophilicity under the conditions used in the study.

### EXAMPLE 2

### Evaluation of the Stability of Nesiritide

The stability of nesiritide at 32° C. under both donor and receptor conditions was evaluated.

### Stability Under Receptor Conditions (Recovery):

The peptide was prepared in various buffer systems to select the optimal solution for use in the receptor. The detergent dodecyltrimethylammonium, with either bromide (DTAB) or chloride (DTAG) as the counter anion, was included in some buffer solutions to reduce nonspecific losses of nesiritide to surfaces. Bovine serum albumin (BSA) was also utilized, both to prevent nonspecific adsorption and to lessen possible proteolysis. The buffer systems evaluated were imidazole, pH 7.0, 10 mM ionic with 15 mM NaCl, and the same solution also containing either 0.5% detergent or 0.1% BSA. The buffers were compared to nesiritide in HPLC grade water, unbuffered (pH −5) with 15 mM NaCl. The concentration of peptide chosen for the recovery studies approximated the amount expected to accumulate in the receptor compartment during transport. The solutions were then exposed to human epidermis, in test tubes for two hours at 32° C. The epidermis was removed, and the recovery solutions were split into two portions. Each portion was analyzed by either SEC or RP-HPLC using the conditions set forth in the tables below to determine the quantity of added peptide remaining (% recovered), and the formation of degradation products.

Size Exclusion Chromatography (SEC)

| Description | Parameter |
|---|---|
| Column: | Pharmacia Superdex Peptide HR 10/30 |
| Flowrate: | 0.8 mL/min |
| Detection: | absorbance at 214 nm, 258 nm |
| Temperature: | column, ambient; autosampler, 4° C. |
| Injection: | 10–100 μL |
| Solvent: | 30% CH₃CN, 0.2 M NaCl in 100 mM H₃PO₄ (pH 2.0) |
| Retention: | Nesiritide: 23 ± 0.5 min/~1.4 |
| MWₐₚ/kDa | with DTT: 15 ± 0.5 min/~3.1 |
| System: | ThermoSeparations |

RP-HPLC: for Degradation Studies

| Description | Parameter |
|---|---|
| Flowrate: | 1.5 mL/min |
| Detection: | millivolt or absorbance at 210 nm (some at 258 nm) |
| Temperature: | column, ambient; autosampler, 4° C. |
| Injection: | 50 μL |
| Gradient: | 10–60% B in 10 min, 60–90% B in 1 min, then hold at |
| | 90% B for 1 min, return 90–10 B in 1 min, hold 4 min |
| | at 10% B |
| Solvents: | A: 0.1% TFA (milliQ water); B: 0.093% TFA (acetonitrile) |
| Time: | 6.8 ± 1.0 min; (DTT reduced Nesiritide: 4.9 ± 0.5 min) |
| System: | Shimadzu or ThermoSeparations |

### Stability Under Donor Conditions:

The peptide was incubated in various buffer systems, both with and without human epidermis, at concentrations to be used for donor solutions. Experiments were conducted at 32° C. for 8 hours and 24 hours. The solutions were separated into portions and analyzed by RP-HPLC using the conditions described in the table below, both directly and after dilution to give a concentration of nesiritide at ˜100 μg/mL. In some cases, donor solutions were also removed and analyzed following ET Flux experiments.

RP-HPLC: for Receptor Analyses

| Description | Parameter |
|---|---|
| Flowrate: | 1.5 mL/min |
| Detection: | millivolt or absorbance at 210 nm (some at 258 nm) |
| Temperature: | column, ambient; autosampler, 4° C. |
| Injection: | 50 μL |
| Gradient: | 10–60% B in 10 min, 60–90% B in 1 min, then hold at |
| | 90% B For 1 min, return 90–10 B in 1 min, hold 4 min |
| | at 10% B |
| Solvents: | A: 0.1% TFA (milliQ water); B: 0.093% TFA (acetonitrile) |
| Time: | 6.8 ± 1.0 min; (DTT reduced Nesiritide: 4.9 ± 0.5 min) |
| System: | Shimadzu or ThermoSeparations |

The peptide exhibited considerable stability at pH 6, 7 or 8 when incubated without human epidermis at 32° C., for an 8-hour period. Samples at pH 6 and 7 remained stable even after 24-hour incubation at 32° C., but the solution at pH 8 displayed considerable loss of nesiritide with apparent disulfide instability. Nesiritide was also tested at pH 7 for a 24-hour period at 32° C., with added human skin, and exhibited no destruction. Analysis of selected donor solutions following ET flux also showed no peptide degradation or change in the disulfide bond.

Nesiritide was quite resistant to destruction following exposure to human epidermis. The degradation of nesiritide was shown to be minimal at pH 6 or 7, even after 24 hours at 32° C. The peptide remained monomeric at the highest concentration analyzed, 3 mM; 10 mg/mL at pH 7 in 10 mM ionic buffer.

### EXAMPLE 3

### Sedimentation Equilibrium Analytical Ultracentrifugation (XLA) of Nesiritide

Nesiritide's tendency towards self-association was assessed using sedimentation equilibrium analytical ultracentrifugation (XLA). An increase in the concentration of peptide in the donor formulation usually is expected to produce an increase in the rate of transport. With many peptides, raising the peptide concentration in solution will also heighten any tendency toward self-association. The conditions of the donor formulation are selected to maximize delivery and minimize aggregation. The development of peptide aggregates in solution can be determined directly by analytical ultracentrifugation. The sensitivity of detection requires at least 5% of the peptide exist as an aggregate.

Solutions of nesiritide at pH 6 and 7, (10 mM ionic imidazole) and pH 8 (10 mM ionic serinamide) were analyzed by sedimentation equilibrium ultracentrifugation. Samples were centrifuged to equilibrium at 44,000 rpm at 32° C., overnight. Absorbance as a function of radial position was determined at 258 nm.

There was no indication of self-association under all conditions tested, by ultracentrifugation, as detailed in the table below. When a highly charged peptide, such as nesiritide at pH 7, is formulated in a low ionic strength buffer, it will behave in a nonideal manner. Under such conditions, the apparent buoyant molecular weight will be lowered because of charge-charge repulsion. Theory has not been developed to estimate the magnitude of this effect when the bulk of the solution ionic strength arises from the peptide and its counter ions (as opposed to the case where the buffer ions contribute the majority of the ionic strength). The calculated value for M(1−v_{bar}*ρ), given in the table below, was based on the behavior expected under ideal conditions.

Sedimentation Equilibrium Ultracentrifugation of Nesiritide
32° C., 44,000 rpm, 258 nm

| | | M(1 − v_{bar} * p) | | |
|---|---|---|---|---|
| Nesiritide | pH of Buffer | (v_{bar} = 0.724 pH 7; M | | |
| mg/mL | (all 10 mM | 3466) | | Residual |
| (~mM) | ionic strength) | calculated | observed | Pattern |
| 3 | 6 (imidazole) | 968 | 630 | nonideal |
| (~1) | | | | |
| | 7 (imidazole) | 968 | 644 | flat |
| | 8 (serinamide) | 954 | 884 | polydisperse |
| 10 | 7 (imidazole) | 968 | 703 | nonideal |
| (~3) | | | | |

### EXAMPLE 4

### Circular Dichroic Spectrometry (CD) of Nesiritide

Circular dichroic spectrometry was used to assess the amount of secondary structure present in nesiritide under aqueous, low ionic strength conditions. In addition, changes in the value calculated for mean residual ellipticity were analyzed to determine if secondary structure, specifically α-helix, could be induced upon the addition of 2,2,2-trifluoroethanol (TFE) or high salt, or both, to nesiritide in aqueous solution at 0.8 mg/mL.

Several methods have been used to estimate the secondary structural components of proteins from the CD spectra. The techniques have been applied to peptide spectra with varying degrees of success (Sreerama, N., Woody, R. W. A Self-Consistent method for the Analysis of Protein Secondary Structure from Circular Dichroism, Analytical Biochemistry, 1993; 209 32-44; Yang, J. T. et al. Separation and Analysis of Peptides and Proteins, Methods Enzymology, 1986; 130 208-269). Computer programs were used to analyze the data from CD scans. Mean residue ellipticity ([θ]nm) was computed at each wavelength and compared to reference spectra to estimate the relative amounts of helix, sheet, turn and aperiodic secondary structure. The spectra for the solution (40% TFE) were also analyzed by fitting summed gaussians to calculate the approximate α-helix and β-sheet components best fitting this data (Holladay, L. A. 1995; personal communications).

The secondary structure assumed by nesiritide in an aqueous environment and induced by the addition of TFE, NaCl or both, were analyzed by CD. The peptide was studied at both 32 and 20° C. The peptide was studied at two concentrations: 0.2 mg/mL (20° C.) to allow data to be obtained at wavelengths below 200 nm, and at 0.8 mg/mL (32° C.) to increase any tendency toward self association. The CD ellipticity values at 222 nm have been interpreted to best reflect the amount of α-helix. The fully helical peptide or protein would have [θ]₂₂₂ₙₘ of −32,000° cm², while the peptide lacking secondary structure would have a value of ˜2400° cm².

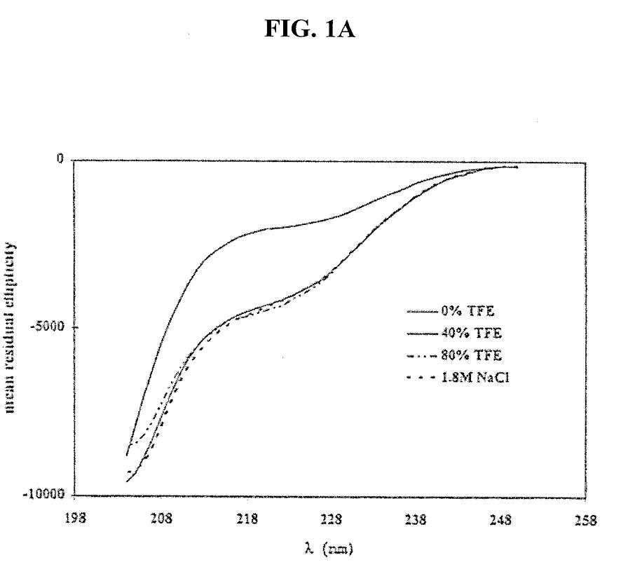

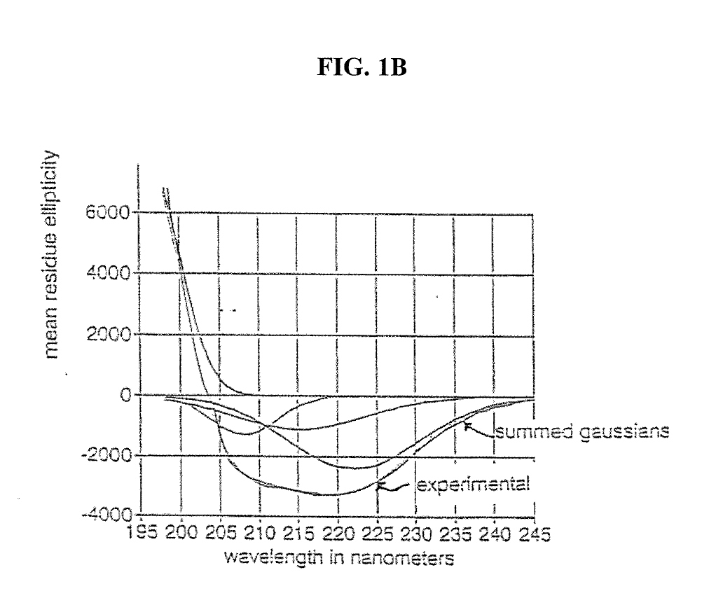

Nesiritide in solution at 0.8 mg/mL (1 mm path) at pH 5.5 (unbuffered, in water), as evaluated by CD, demonstrated [θ]₂₂₂ₙₘ of −1984 deg cm² dmol⁻¹, which suggested essentially no secondary structure. At 32° C., the inclusion of up to 80% TFE, or up to 1.8 M NaCl, or a combination of the two, did not induce any significant α-helix with a solution of nesiritide (FIG. 1A). The data in FIG. 1A, and the value for [θ]₂₂₂ₙₘ (˜3200° cm² dmol⁻¹), suggest that a small amount of secondary structure may be inducible, and the amount is the same whether 40% or 80% TFE or 1.8 M NaCl is used.

At 20° C., nesiritide at ˜0.2 mg/mL in pH 7 cacodylate buffer at 10 mM ionic, also showed a small amount of inducible α-helix at 40% TFE (FIG. 1B). The use of the lower concentration, with a 2 mm path, allowed data to be collected down to 197 nm. The difference spectrum (FIG. 1B) was resolved into component gaussian bands (Holladay, L. A., Savage, C. R., Cohen, S., Puett, D. Biochemistry, 1976; 15 2624-2633; Holladay, L. A., Hammonds, R. G., Jr., Puett, D. Biochemistry, 1974; 13 1653-1661) in order to estimate the amount of α-helix and β-sheet formed. Estimates of secondary structure were made by computing the ratio of the observed rotational strength (R cgs) with those for poly-L lysine in either the α-helical or β-sheet form. The table below gives the parameters of the resolved gaussians, and the estimates of secondary structure. Nesiritide in 40% TFE probably exists as a mixture of conformers.

Gaussian Ban Parameters Used for 40% TFE Difference Spectrum
Nesiritide 0.2 mg/mL, 20° C., pH 7, 10 mM Ionic Cacodylate, 2 mm Path

| | α-Helix | β-Sheet | α-Helix | Helix/Sheet |
|---|---|---|---|---|
| Assignment | n → Π* | n → Π* | Π → Π* | Π → Π* |
| Wavelength | 222 | 215 | 208 | 195 |
| (nm) | | | | |
| [θ]₀ | −2400 | −1100 | −1300 | 8000 |
| (peak | | | | |
| ellipticity) | | | | |
| Δ (peak | 12 | 12 | 6 | 5.5 |
| half width) | | | | |
| R | −7.6 × 10⁻⁴¹ | −1.6 × 10⁻⁴⁰ | −4.6 × 10⁻⁴¹ | |
| % Structure | 6.4 | 5.1 | 5.0 | not determined |
| Estimate | | | | |

The value calculated for the free energy of adsorption to the HIC column, δΔ G_{ads} was 0.46 k J mole⁻¹, in imidazole at pH 7, 10 mM ionic, which indicated a hydrophilic peptide.

### EXAMPLE 5

### In Vitro Electrotransport of Nesiritide—Initial Studies

Nesiritide was screened in vitro for its electrotransport (ET) properties with heat-separated human epidermis/stratum corneum (skin).

In vitro electrotransport of nesiritide across heat-separated human epidermis/stratum corneum was conducted using anodic drive with ET cells in three separate sets of experiments, using human epidermis from three sources under the conditions set forth in the table below.

Conditions for In Vitro Electrotransport

| Description | Parameter |
|---|---|
| Cells (ECs): | Custom-built ETcells |
| Current density: | 100 μA/cm² |
| Current control: | Potentiostat/Galvanostat; Digital Multimeter |
| Temperature: | 32° C. Incubator |
| Test Membrane | a) surgical: 58 y o female breast, white |
| human epidermis | b) fast frozen: 39 y o male abdominal, white |
| | c) cadaver: 53 y o female thigh, white |
| Donor: | 1 mM or 3 mM Nesiritide pH 7.0, imidazole, 10 mM |
| | ionic |
| Receptor: | pH 7.0, imidazole, 10 mM ionic with 15 mM NaCl, |
| | 0.5% DTAC or DTAB |

The pH of donor solutions was monitored before and after the experiments. The amount of passive transport was determined in fully assembled ET cells, incubated at 32° C. for 1 hour, with subsequent analysis of the receptor solution by RP-HPLC. Results from any ET cell that showed passive transport (most likely indicating an epidermis which was leaking) were not used. The applied constant current was 100 μA/cm². The receptor solution was removed for analysis and replenished at 2 hour intervals during the first eight hours of testing, and then after 24 hours and 26 hours. The voltage drop across each cell was monitored at intervals throughout the experiment. Additionally, after the completion of transport studies, the solutions from some donor compartments were removed and analyzed either for evidence of peptide degradation or for pH changes.

When comparing data obtained using different conditions during ET Flux, it is thought to be more useful to compare the median values for each case (Holladay, L. A. 1995; personal communications). The use of the median as a figure of merit for ET flux data decreases the impact of both measurement and recovery problems for cells with low transport values. It also minimizes the impact of data from ECs with higher values, which may represent skin that was partially leaking. In order to compare the values of the median from different studies, it is necessary to have an estimate of the confidence interval for each median. If the values for the medians are different, but the confidence intervals overlap, then the results are not truly distinguished. For this work with nesiritide, a Bootstrap method (Diciccio, T. J., Romano, J. P. A Review of Bootstrap Confidence Intervals, J. R. Statist Soc B, 1988; 50 338-354; Efron, B., Tibshirani, R. Bootstrap Methods for Standard errors, Confidence Intervals, and Other Measures of Statistical Accuracy, Statistical Science, 1986; 1 54-77) was used to determine 95% and 67% confidence intervals for the data sets compared. The method estimates the intervals using considerable amounts of computation; 32,000 trials of the algorithm were used for these analyses.

The results from three studies are shown in the table below.

Electrotransport of Nesiritide (1 mM and 3 mM): 8 hour Delivery
Fresh and Frozen Skin

| Study | a & c | c | a & c | b | b |
|---|---|---|---|---|---|
| Skin Type | Fresh | | | Frozen | |
| Donor | 1 mM | 3 mM | combine | 1 mM | 3 mM |
| Nesiritide | | | 1 mM & | | |
| | | | 3 mM | | |
| Mean: | 3.0 | 8.2 | 4.7 | 10.7 | 24.9 |
| μg cm⁻² h⁻¹ | | | | | |
| Standard | 4.7 | 13.4 | 9.3 | 10.7 | 28.9 |
| Deviation | | | | | |
| Median | 1.6 | 2.0 | 1.6 | 6.3 | 5.6 |
| μg cm⁻² h⁻¹ | | | | | |
| 95% | 1.1–2.1 | 1.2–4.7 | 1.2–2.1 | 3.5–14.4 | 3.3–41.3 |
| Confidence | | | | | |
| Interval | | | | | |
| 67% | 1.4–1.9 | 1.4–2.8 | 1.5–2.0 | 5.5–12.2 | 3.5–25.3 |
| Confidence | | | | | |
| Interval | | | | | |
| Number of | 58 | 28 | 86 | 19 | 24 |
| Data Points | | | | | |

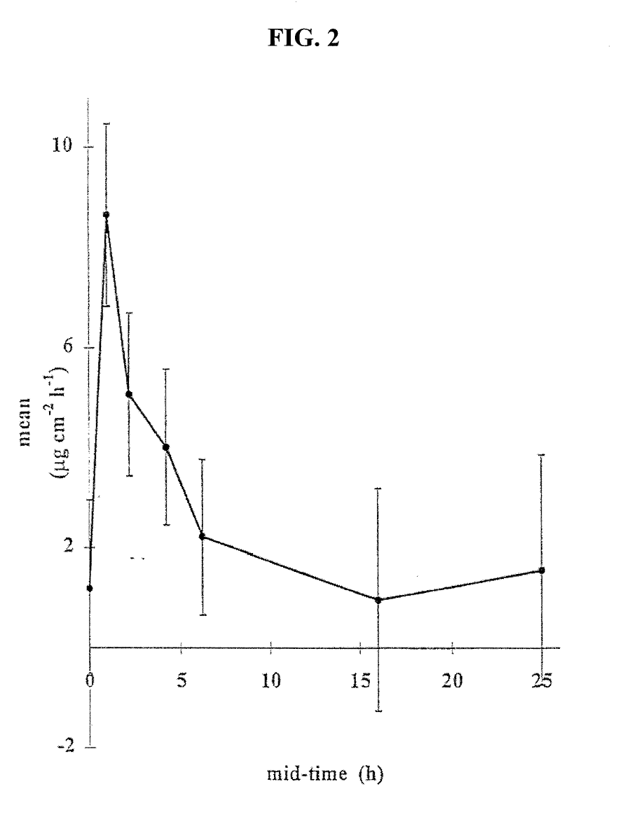

Evaluation of these results indicated that there was a significant difference between the studies using frozen skin and those using fresh skin. As a consequence, the data from study b (frozen skin) were excluded from the final summary. The results of transport with respect to time (shown in the table below and in FIG. 2) showed a decline in transport, which approaches zero transport by 24 hours. The data obtained from samplings at 24 hours (mid-time 16 hours) and 26 hours (mid-time 25 hours) were not included in the final calculation of mean and median transport.

Electrotransport of Nesiritide (1 mM and 3 mM)
8 hour and 24 hour Delivery

| Study | a & c | c | b | b |
|---|---|---|---|---|
| Sampling | up to 8 h | 8 h–26 h | up to 8 h | 8 h–26 h |
| Duration | | | | |
| Skin Type | fresh | fresh | frozen | frozen |
| Mean: | 4.7 | 1.3 | 18.6 | 1.5 |
| μg cm⁻² h⁻¹ | | | | |
| Standard | 1.0 | 0.5 | 3.6 | 0.6 |
| Error | | | | |
| of the Mean | | | | |
| Median | 1.6 | 0.5 | 6.3 | 0.9 |
| μg cm² h⁻¹ | | | | |
| 95% | 1.2–2.1 | 0.1–1.0 | 3.5–14.4 | 0.4–1.3 |
| Confidence | | | | |
| Interval | | | | |
| 67% | 1.5–2.0 | 0.4–0.7 | 5.2–10.1 | 0.4–1.0 |
| Confidence | | | | |
| Interval | | | | |
| Number of | 86 | 20 | 43 | 15 |
| Data Points | | | | |

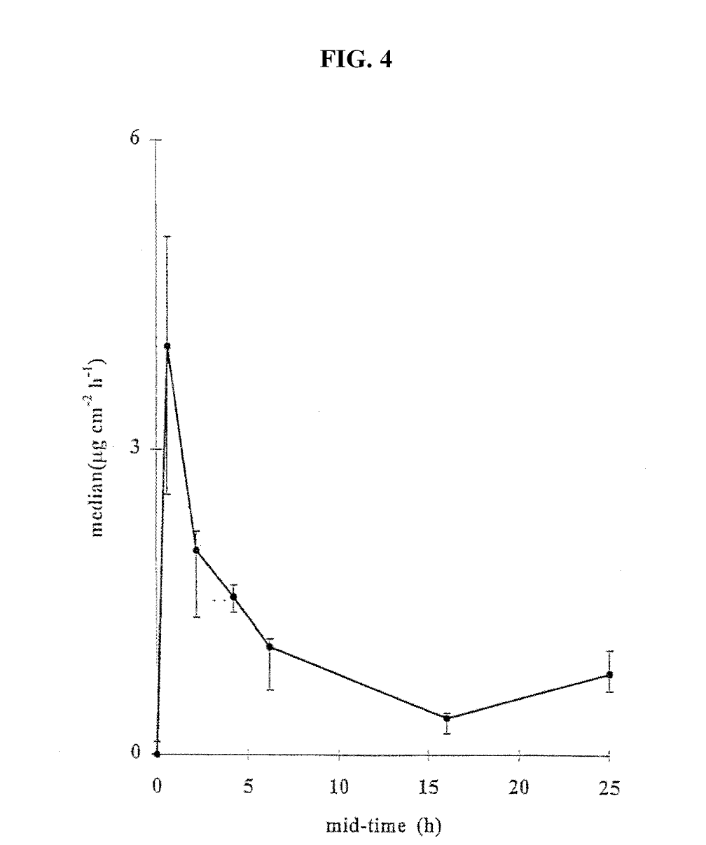

The decrease in mean transport with time, as shown in FIG. 2, was plotted with the error expressed in standard error of the mean (computed from the standard deviation, divided by the square root of the number of data points). At the later time points, mid-time 16 hours and 2 hours, it was not clear if the values were actually significantly above zero. As an alternative method to present the data, in FIG. 4, the median ET Flux with respect to time was plotted. The error bars shown are the 67% confidence limits, as estimated by the Bootstrap method, and detailed in the table below.

Median Nesiritide Flux: Fresh Skin

| | Median | | | | |
|---|---|---|---|---|---|
| | Transport | Bootstrap Confidence Interval | | | |
| Mid-time | (μg cm⁻² h⁻¹) | +95% | +67% | −67% | −95% |
| 0 | 0 | 2.83 | 0.13 | 0^{a} | 0^{a} |
| 0.6 | 4.00 | 6.11 | 5.06 | 2.56 | 2.17 |
| 2.2 | 2.01 | 2.86 | 2.20 | 1.35 | 1.11 |
| 4.2 | 1.55 | 2.03 | 1.67 | 1.40 | 0.85 |
| 6.2 | 1.06 | 1.49 | 1.14 | 0.64 | 0.42 |
| 16 | 0.37 | 1.47 | 0.42 | 0.22 | 0.12 |
| 25 | 0.80 | 1.22 | 1.03 | 0.63 | 0^{a} |

^{a}method will not produce negative numbers

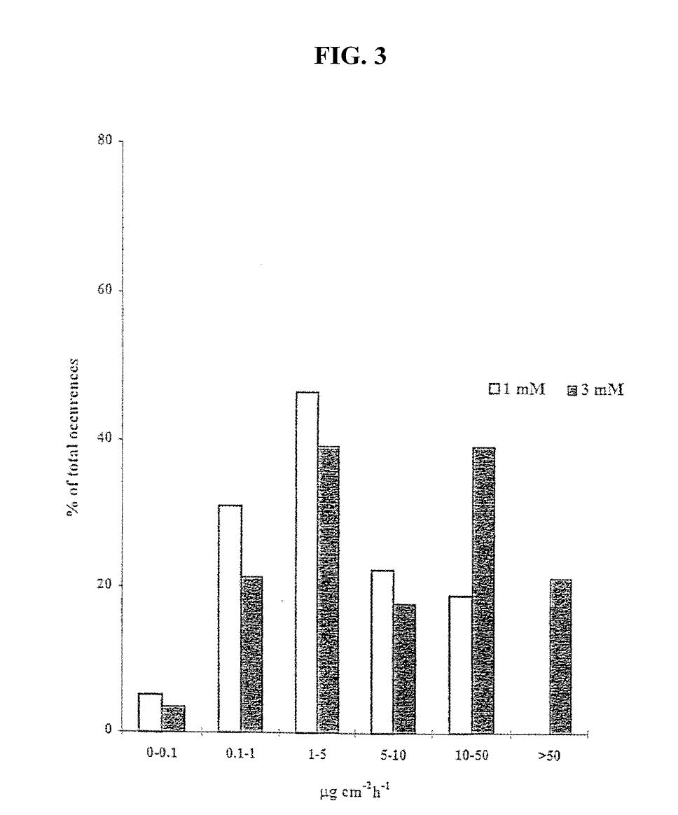

When the data obtained from ET Flux experiments under varied conditions were compared, the median was thought to be the more useful value (Holladay, L. A. 1995; personal communications). The median has been shown to be less impacted by both the measurement and the recovery problems, which are maximal in samples from ET cells with lower transport. It also minimized the intensity of results from ET cells with high values, which could reflect a slightly leaky epidermis. The median values for transport, whether the donor contained 1 mM or 3 mM nesiritide, were not significantly different (FIG. 3), and were combined to compute the median and mean. The comprehensive median value for transport of nesiritide through human skin was 1.6 μg cm⁻²h⁻¹, with a 95% confidence interval of 1.2-2.1.

### EXAMPLE 6

### Preparation of Nesiritide Hydrogels

Hydrogels were typically prepared by dissolving polyvinyl alcohol (PVOH) at 19 wt % in purified water at 90° C. for 30 minutes, dispensing the gel solution into disks, and freezing overnight at about −20° C. The grade of PVOH used had a viscosity of 28 MPa·s (for a 4% aqueous solution at 20° C.). The formed hydrogels were then allowed to imbibe nesiritide as a concentrated aqueous solution at room temperature to obtain the desired nesiritide loading. Alternatively, nesiritide loading was achieved by adding nesiritide to the PVOH hydrogel solution before freezing. In the thermally processed formulations, PVOH was dissolved in purified water at 90° C. as described above. After reduction of the temperature to 50° C., an aqueous solution of nesiritide was added to the PVOH solution and allowed to mix for 30 minutes. The PVOH-ROH-nesiritide mixture was dispensed into disks and freeze-cured. Finished hydrogels were used in flux studies or extracted with purified water for drug-stability analysis.

### EXAMPLE 7

### In Vitro Electrotransport of Nesiritide Using Hydrogel Reservoirs

In vitro electrotransport flux experiments were conducted using either synthetic polymeric Nulepore membranes or heat separated human cadaver epidermis. Custom-built horizontal diffusion cells were used for all in vitro skin flux experiments. Silver and silver chloride electrodes were used to apply current across the cell. Nesiritide hydrogels were placed between the silver anode and human heat-separated epidermis. In addition, a PVOH hydrogel containing a polymeric chloride source was placed between the silver anode and the drug hydrogel. A 1/10 dilution of Dulbecco's phosphate buffered saline (0.015 M NaCl) served as the receptor solution, which was continuously pumped through the receptor compartment. At multiple time points, receptor samples for HPLC analysis were collected using a custom-built, automated Hanson Research Microette™ collection system.

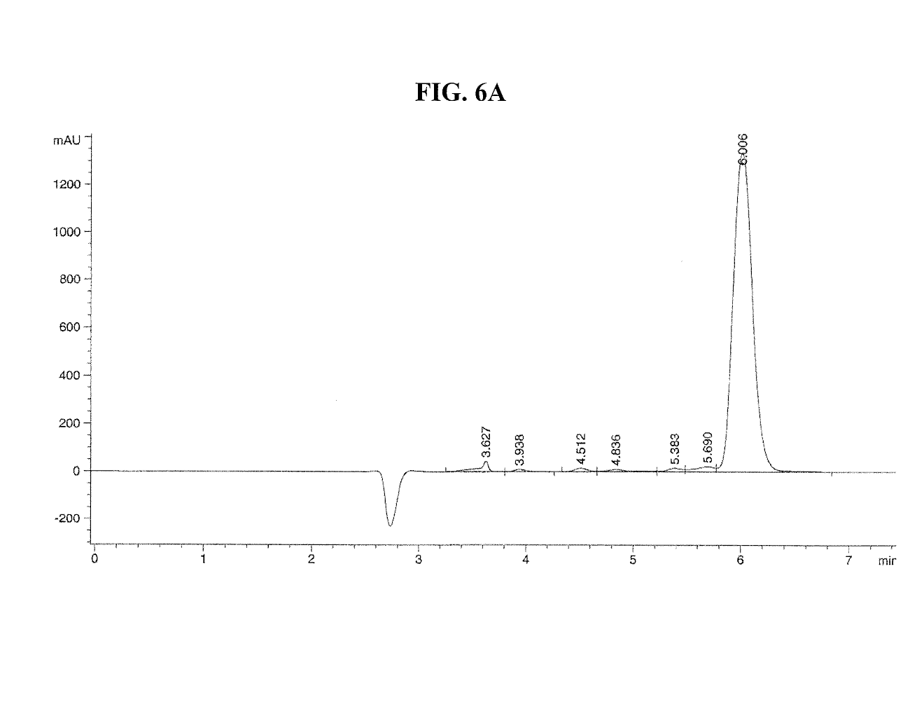

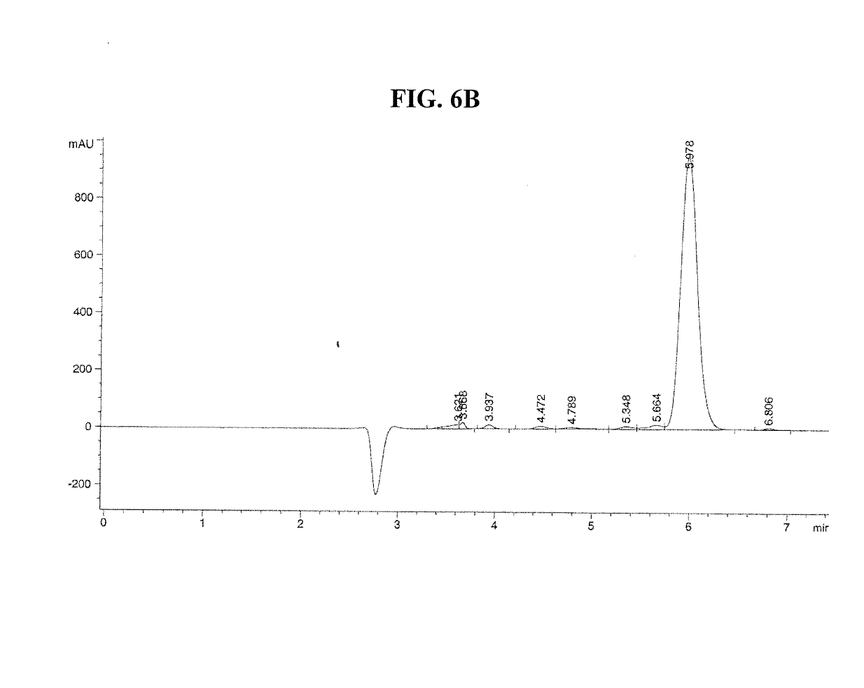

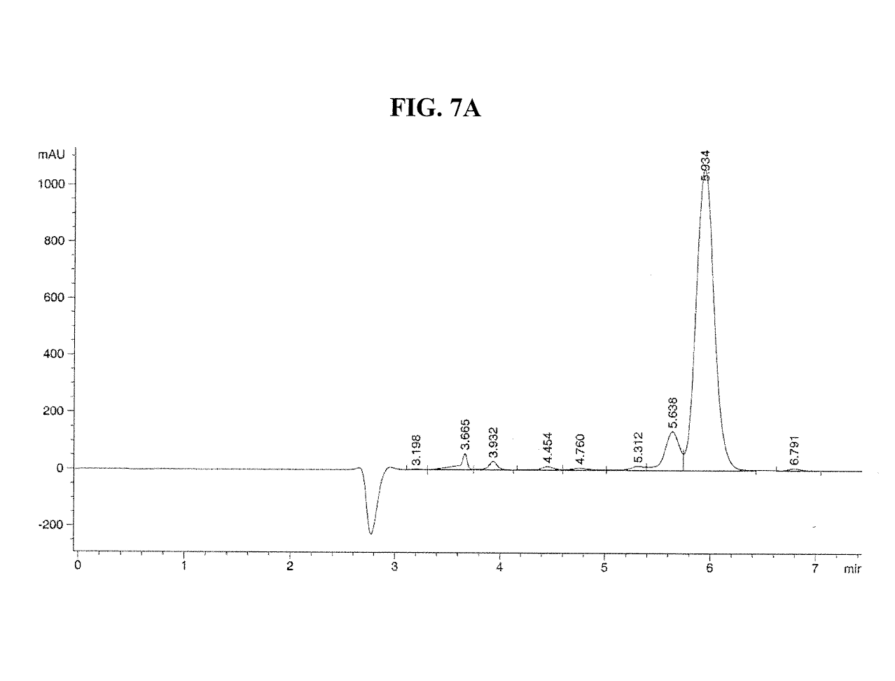

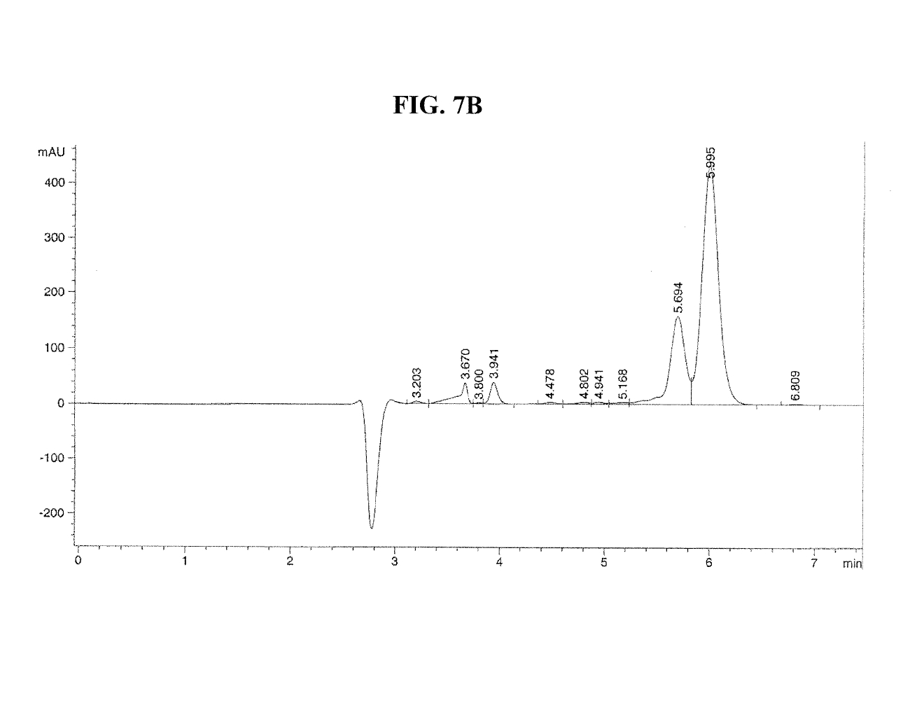

The receptor samples were analyzed for nesiritide using a HPLC assay. Flux data was plotted as μg/cm² h versus time as shown in FIG. 5. In addition, as show in FIGS. 6B and 7B, formulations were also extracted for nesiritide and analyzed via a HPLC assay after use in a flux experiment. A few samples of unused formulations were also analyzed for nesiritide as controls, as shown in FIGS. 6A and 7A. HPLC analysis of used versus unused nesiritide formulations provided an estimate of the “in use” stability of the drug under electrotransport conditions and demonstrated that, under typical electrotransport conditions (hydrogel formulation, over 24 hours, at 0.1 mA/cm² and 32° C.) nesiritide showed sufficient stability.

The entire disclosure of each patent, patent application, and publication cited or described in this document is hereby incorporated herein by reference.

We claim:

1. A method for the transdermal administration by electrotransport of nesiritide or a pharmaceutically acceptable salt thereof to a patient in need thereof comprising
providing a device for the electrotransport delivery of nesiritide comprising
a donor electrode assembly comprising a donor reservoir that comprises a matrix containing nesiritide;
a counter electrode assembly; and
a source of electrical power that is connected to the donor and counter electrode assemblies; and
administering the nesiritide to the patient at a therapeutically effective dose using the device.

2. The method of claim 1 wherein the nesiritide is administered to the patient continuously, intermittently, in a bolus dose, or in a bolus dose followed by continuously.

3. The method of claim 2 wherein the nesiritide is administered to the patient continuously at a dose of 0.500 μg/kg/min to 0.05 μg/kg/min.

4. The method of claim 2 wherein a bolus dose of 2 μg/kg/min of nesiride is administered to the patient followed by a continuous dose of 0.01 μg/kg/min.

5. The method of claim 1 wherein the device further comprises a sensor that monitors the patient's blood pressure, and the output of the electrical power source is automatically adjusted in accordance with changes in the patient's blood pressure.

6. The method of claim 1 wherein the matrix that comprises the donor reservoir is a polymeric matrix comprising a soluble or insoluble hydrophilic polymer, a gel matrix comprising a hydrophilic polymer that swells when exposed to water, or a polymer electrolyte matrix.

7. The method of claim 1 wherein the source of electrical power delivers a direct current, a pulsed current, or an alternating reverse polarity current.

8. A device for the transdermal administration by electrotransport of nesiritide or a pharmaceutically acceptable salt thereof to a patient in need thereof comprising
a donor electrode assembly comprising a donor reservoir that comprises a matrix containing nesiritide;
a counter electrode assembly; and
a source of electrical power that is connected to the donor and counter electrode assemblies.

9. The device of claim 8 wherein the nesiritide is administered continuously, intermittently, in a bolus dose, or in a bolus dose followed by continuously.

10. The device of claim 9 wherein the nesiritide is administered to the patient continuously at a dose of 0.500 μg/kg/min to 0.05 μg/kg/min.

11. The device of claim 9 wherein a bolus dose of 2 μg/kg/min of nesiride is administered to the patient followed by a continuous dose of 0.01 μg/kg/min.

12. The device of claim 8 wherein a bolus dose of 2 μg/kg/min of nesiride is administered to the patient followed by administration of a continuous dose of 0.01 μg/kg/min.

13. The device of claim 8 wherein the matrix that comprises the donor reservoir is a polymeric matrix comprising a soluble or insoluble hydrophilic polymer, a gel matrix comprising a hydrophilic polymer that swells when exposed to water, or a polymer electrolyte matrix.

14. The device of claim 8 wherein the source of electrical power delivers a direct current, a pulsed current, or an alternating reverse polarity current.

15. A method for treating congestive heart failure consisting essentially of transdermally administering nesiritide or a pharmaceutically acceptable salt thereof to a patient suffering from congestive heart failure using an electrotransport device comprising
a donor electrode assembly comprising a donor reservoir that comprises a matrix containing nesiritide;
a counter electrode assembly; and
a source of electrical power that is connected to the donor and counter electrode assemblies.

16. The method of claim 15 wherein the nesiritide is administered continuously, intermittently, in a bolus dose, or in a bolus dose followed by continuously.

17. The method of claim 16 wherein the nesiritide is administered to the patient continuously at a dose of 0.500 μg/kg/min to 0.05 μg/kg/min.

18. The method of claim 16 wherein a bolus dose of 2 μg/kg/min of nesiride is administered to the patient followed by a continuous dose of 0.01 μg/kg/min.

19. The method of claim 15 wherein a bolus dose of 2 μg/kg/min of nesiride is administered to the patient followed by administration of a continuous dose of 0.01 μg/kg/min.

20. The method of claim 15 wherein the matrix that comprises the donor reservoir is a polymeric matrix comprising a soluble or insoluble hydrophilic polymer, a gel matrix comprising a hydrophilic polymer that swells when exposed to water, or a polymer electrolyte matrix.

21. The method of claim 15 wherein the source of electrical power delivers a direct current, a pulsed current, or an alternating reverse polarity current.
